(19) United States
(12) Patent Application Publication
(10) Pub. No.: US 2009/0209824 A1
(43) Pub. Date: Aug. 20, 2009

(54) OCT OPTICAL PROBE AND OPTICAL TOMOGRAPHY IMAGING APPARATUS

(75) Inventor: Yutaka Korogi, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignees: FUJIFILM Corporation, Tokyo (JP); Fujinon Corporation, Saitama-shi (JP)

(21) Appl. No.: 12/368,084

(22) Filed: Feb. 9, 2009

(30) Foreign Application Priority Data
Feb. 14, 2008 (JP) 2008-032555

(51) Int. Cl. A61B 1/06

(52) U.S. Cl. 600/160

(57) ABSTRACT

Safe and inexpensive visual confirmation of the scanning position of an OCT optical probe, which is inserted into a forceps channel of an endoscope, is provided. The OCT optical probe to be inserted into a forceps channel of an endoscope that applies illumination light to a site to be observed in a body cavity and images reflected light from the site to be observed, includes: a substantially cylindrical and long sheath to be inserted into the body cavity; a distal end optical system disposed in the sheath and being rotatable about the longitudinal axis of the sheath; and a ferrule integrally fixed to the distal end optical system, wherein the ferrule includes a reflecting section adapted to reflect a part of the illumination light toward the endoscope.

### BACKGROUND OF THE INVENTION

1. Field of the Invention

The present invention relates to an OCT optical probe which is inserted into a forceps channel of an endoscope, and an optical tomography imaging apparatus which employs the OCT optical probe to acquire an optical tomographic image.

2. Description of the Related Art

Conventionally, for acquiring tomographic images within a body cavity, optical tomography imaging apparatuses using OCT measurement techniques have sometimes been used. In such an optical tomography imaging apparatus, low-coherent light emitted from a light source is divided into measurement light and reference light. The measurement light is applied to a subject to be measured, and then reflected light from the subject to be measured is combined with the reference light. Then, a tomographic image is acquired based on intensity of interference light formed between the combined reflected light and reference light. The OCT measurement techniques are classified into TD (Time Domain)—OCT measurement techniques and FD (Fourier Domain)—OCT measurement techniques. Recently, the FD-OCT measurement is attracting attention because of its ability to provide high-speed measurement. Typical systems that carry out the FD-OCT measurement include an SD (Spectral Domain)—OCT system and an SS (Swept Source)—OCT system.

The optical tomography imaging apparatuses use an OCT optical probe, which is inserted into a body cavity and guides and moves the measurement light to scan at least in one-dimensional direction, and guides the reflected light to acquire an optical tomographic image of a subject to be measured. The OCT optical probe may be inserted through a forceps channel of an endoscope which applies illumination light to a site to be observed and images the site to be observed. Usually, this type of OCT optical probe is used in a state where it protrudes from the distal end of the endoscope by a length of about 30 mm to about 60 mm. This allows the operator to view the OCT optical probe in the body cavity using the endoscope, thereby improving safety during measurement.

Further, the operator needs to visually confirm the scanning position of the OCT optical probe in order to reduce burden on the subject by efficiently acquiring the optical tomographic image of a desired site. It has conventionally been known to form the sheath of the OCT optical probe by a colored sheath and a transparent sheath, as shown in FIGS. 9A and 9B, to allow visual confirmation of the scanning position of the OCT optical probe. Namely, as shown in FIGS. 9A and 9B, a part of the OCT optical probe at the proximal end side is covered with the colored sheath, to which a colorant such as carbon black is applied, and a part of OCT optical probe at the distal end side is covered with the transparent sheath via an adaptor made of stainless steel, for example, so that the operator can visually confirm the area around the scanning position of the OCT optical probe based on the position of the transparent sheath. Further, U.S. Pat. No. 6,668,185 has proposed a technique for allowing visual confirmation of the scanning position of the OCT optical probe by the operator, in which visible light, such as He—Ne laser light, serving as aiming light is superimposed on the measurement light of the OCT optical probe coaxially with measurement light, so that the scanning position is displayed as a bright spot.

In the approaches shown in FIGS. 9A and 9B, however, it is necessary to consider biocompatibility of the applied colorant in view of safety. In addition, in the approach shown in FIG. 9A, since a ferrule, which is integrally fixed to a distal end optical system for moving the measurement light to scan, is not fixed to the sheath, the distal end optical system moves within the sheath in the direction of arrow A due to flexure of the OCT optical probe during operation. Therefore, the confirmation of the area around the scanning position based on the position of the transparent sheath is affected by the flexure of the OCT optical probe. On the other hand, in the approach shown in FIG. 9B, the ferrule is fixed to the adaptor made of stainless steel, or the like, and therefore the scanning position within the sheath is fixed. However, an optical fiber held by the ferrule for guiding the measurement light and the reflected light may be stressed due to the flexure of the OCT optical probe.

Although the technique disclosed in U.S. Pat. No. 6,668,185 is free of the influences of the biocompatibility of the colorant and the flexure of the OCT optical probe, superimposing the aiming light on the measurement light coaxially with the measurement light requires provision of additional optical elements, such as a dichroic mirror and a coupler for combining light, along the optical path length, and this will lead to a cost increase.

### SUMMARY OF THE INVENTION

In order to address the above-described problems, an aspect of the OCT optical probe of the invention, which is to be inserted into a forceps channel of an endoscope that applies illumination light to a site to be observed in a body cavity and images reflected light from the site to be observed, includes: a substantially cylindrical and long sheath to be inserted into the body cavity; a distal end optical system disposed in the sheath and being rotatable about the longitudinal axis of the sheath; and a ferrule integrally fixed to the distal end optical system, wherein the ferrule includes a reflecting section adapted to reflect a part of the illumination light toward the endoscope. The term “substantially cylindrical” herein refers to a shape that may not necessarily be strictly cylindrical about a straight axis from one end to the other end, and the sheath may include a gently curved shape, such as a semispherical shape, at the distal end thereof. Further, the cross-sectional shape of the sheath may not necessarily be a mathematically-strict circle, and may be ellipsoidal, or the like. The “part of the illumination light” herein refers to the part of the illumination light emitted from the endoscope which is applied to the ferrule. The “to reflect . . . toward the endoscope” refers to reflecting the part of the illumination light toward an imaging means, such as an imaging lens, provided in the endoscope.

The reflecting section may be formed on a partial or entire area of an outer circumference of the ferrule. The “outer circumference of the ferrule” herein refers to the outer circumference of the ferrule to which the illumination light is applied through the sheath.

The reflecting section may be formed by uneven surfaces provided on the outer circumference of the ferrule. The “uneven surfaces” herein are not limited to those formed by directly machining the outer circumference of the ferrule, and may include those formed by providing a machined member having uneven surfaces on the outer circumference of the ferrule. The uneven surfaces may be formed by protrusions formed on the outer circumference of the ferrule or depressions formed in the outer circumference of the ferrule.

The optical tomography imaging apparatus according to the invention is formed by an optical tomography imaging apparatus using any of the above-described measurement techniques, which employs the OCT optical probe according to the invention. Namely, an aspect of the optical tomography imaging apparatus according to the invention includes: a light source unit for emitting light; a light dividing section for dividing the light emitted from the light source unit into measurement light and reference light; an OCT optical probe for applying the measurement light to a subject to be measured; a combining section for combining the reference light with reflected light from the subject to be measured when the measurement light is applied to the subject to be measured; an interference light detecting unit for detecting interference light formed between the combined reflected light and reference light; and a tomographic image processing unit for detecting reflection intensity at a plurality of depth positions in the subject to be measured based on frequency and intensity of the detected interference light, and acquiring an optical tomographic image of the subject to be measured based on the intensity of the reflected light at each depth position, wherein the OCT optical probe includes the OCT optical probe according to the invention.

### BRIEF DESCRIPTION OF THE DRAWINGS

FIG. 1 is a schematic structural diagram of an optical tomography imaging apparatus 1, to which an OCT optical probe 2 of the invention is applied, an endoscope 50 and a display unit 80,

FIG. 2 is a diagram illustrating a distal end portion 10 of the OCT optical probe 2 of the invention,

FIGS. 3A-3E illustrate further embodiments of a reflecting section 16 of the OCT optical probe 2 of the invention,

FIG. 4 illustrates swept wavelength of light emitted from a light source unit 110,

FIGS. 5A and 5B illustrate a period clock signal generated by a period clock generator unit 120,

FIG. 6 is a block diagram illustrating the schematic configuration of a tomographic image processing unit 150,

FIGS. 7A and 7B illustrate an interference signal IS at an interference signal converting unit 152,

FIG. 8 schematically illustrates an operation carried out by a tomographic information generator unit 154, and

FIGS. 9A and 9B are schematic diagrams illustrating conventional OCT optical probes.

### DESCRIPTION OF THE PREFERRED EMBODIMENTS

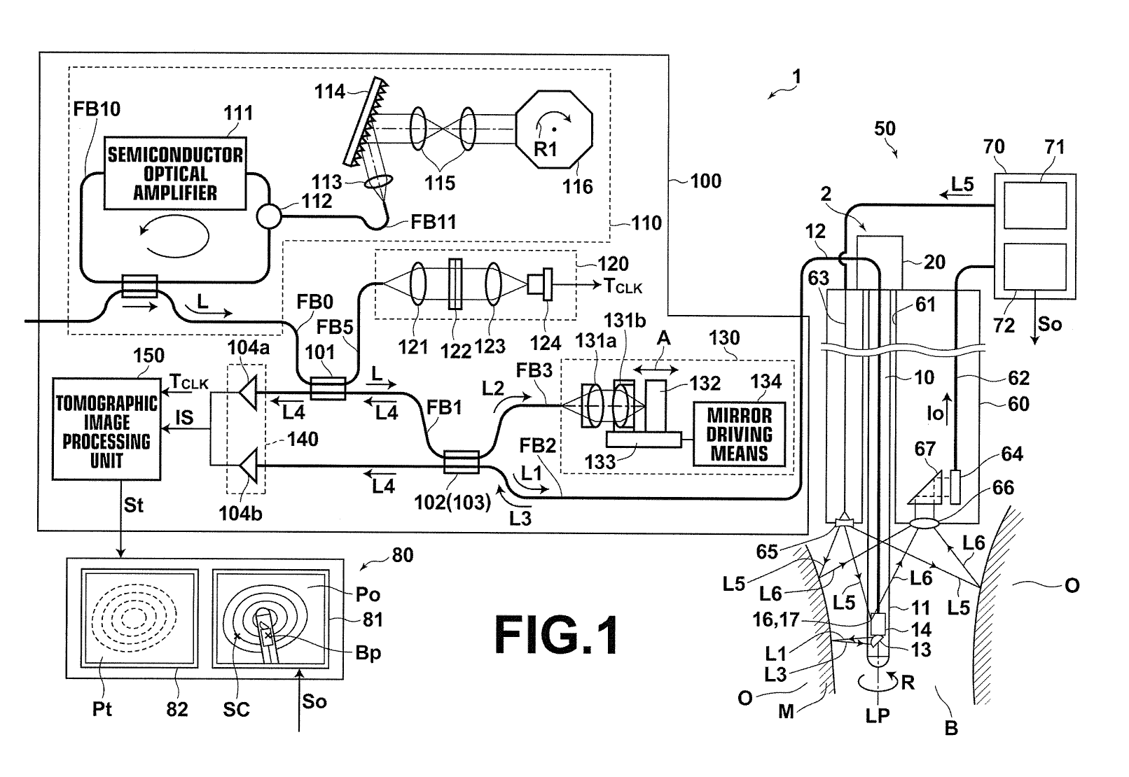

Hereinafter, embodiments of the present invention will be described with reference to the drawings. FIG. 1 is a schematic structural diagram showing an optical tomography imaging apparatus 1, to which OCT optical probe 2 of the invention is applied, an endoscope 50 and a display unit 80.

The endoscope 50 is described. The endoscope 50 includes an inserted portion 60 which is inserted into a body cavity B, and an observation image acquiring unit 70 for acquiring an observation image Po in the body cavity B.

The inserted portion 60 includes a forceps channel 61 that extends through the inserted portion 60, and a CCD cable 62 and a light guide 63, which are built in the inserted portion 60 and extend to the distal end. A CCD image pickup device 64 is connected to the distal end of the CCD cable 62, and an illumination lens 65 is disposed at the distal end of the light guide 63. An imaging lens 66 is disposed at the distal end of the inserted portion 60, and a prism 67 is disposed at an inner position than the imaging lens 66.

In this embodiment, the OCT optical probe 2 is inserted through the forceps channel 61. The light guide 63 directs illumination light L5 emitted from the observation image acquiring unit to the illumination lens 65. The illumination lens 65 emits the illumination light L5 toward the site to be observed O. The imaging lens 66 collects and directs reflected light L6 from the site to be observed O illuminated by the illumination light L5 to the prism 67. The prism 67 reflects the reflected light L6 from the imaging lens 66 so that the reflected light L6 is focused on the CCD image pickup device 64. The CCD image pickup device 64 generates observation image information Io through photoelectric conversion.

The observation image acquiring unit 70 includes an illumination light source 71 and a video processor 72. The illumination light source 71 emits the illumination light L5 to the light guide 63 connected thereto. The video processor 72 carries out processing, such as correlation double sampling, clamping, blanking and amplification, based on observation image information Io inputted from the CCD image pickup device 64 via the CCD cable 62 connected thereto, and outputs an observation image signal So to the display unit 80, which will be described later.

Next, the optical tomography imaging apparatus 1 is described. The optical tomography imaging apparatus 1 includes the OCT optical probe 2, which is inserted through the forceps channel 61 of the endoscope 50 into the body cavity B, and an optical tomography processing unit 100.

The OCT optical probe 2 includes a flexible and long distal end portion 10, a proximal end portion 20 joined to the proximal end of the distal end portion 10, and an optical fiber 12. The distal end portion 10 is inserted through the forceps channel 61 into the body cavity B, and has a length of about 3 m. The proximal end portion 20 contains a driving means (not shown), which drives the optical fiber 12 to rotate in the direction of arrow R to move the measurement light L1 to scan about the optical axis LP. One end of the optical fiber 12 is removably connected to the optical tomography processing unit 100, and the other end of the optical fiber 12 is inserted through the proximal end portion 20 and the distal end portion 10 to extend to an area in the vicinity of the distal end of the distal end portion 10.

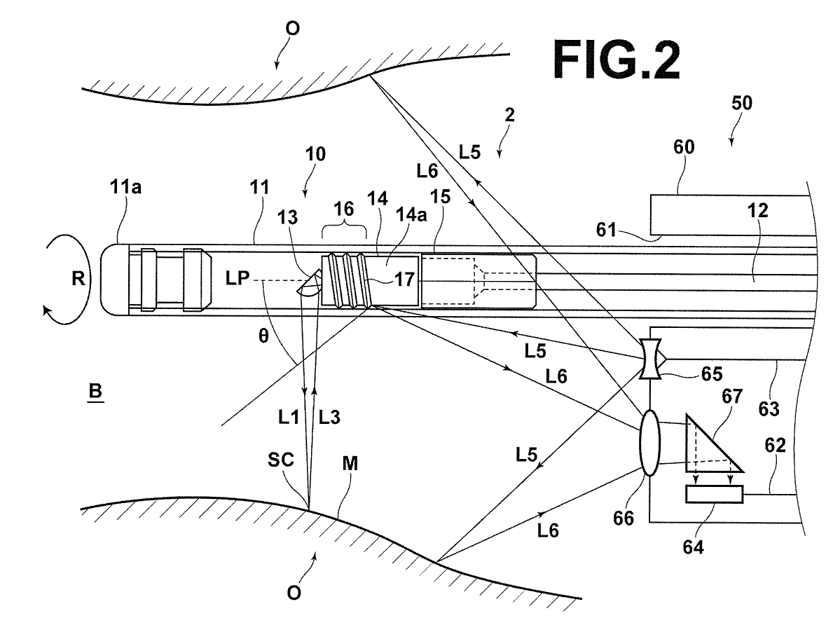

Now, the distal end portion 10 of the OCT optical probe 2 is described in detail. FIG. 2 illustrates the distal end portion 10 of the OCT optical probe 2 of the invention. The distal end portion 10 of the OCT optical probe 2 includes a substantially cylindrical sheath 11, the optical fiber 12 contained in and extending along the longitudinal direction of the sheath 11, a distal end optical system 13 which collects and directs the light emitted from the optical fiber 12 to a subject to be measured M, a ferrule 14 which is integrally fixed to the distal end optical system 13 coaxially with an optical axis LP of the optical fiber 12 and holds the optical fiber 12 via an adhesive, or the like, and a sleeve 15 which fits around the ferrule 14 to reinforce holding the optical fiber 12.

The sheath 11 is formed by a flexible member. In this embodiment, the distal end of the sheath 11 is closed with a cap 11a. The sheath 11 is made of a material that transmits the illumination light L5 applied from the endoscope 50 and the reflected light L6.

The optical fiber 12 is covered with a flexible shaft (not shown), which is formed by a closed coil spring of a metal wire that is closely wound in a spiral form. The optical fiber 12 may be fixed to the flexible shaft.

The distal end optical system 13 has a substantially spherical shape. The distal optical system 13 deflects the measurement light L1 emitted from the optical fiber 12 and collects and directs the measurement light L1 to the subject to be measured M. The distal optical system 13 also deflects the reflected light L3 from the subject to be measured M and collects and directs the reflected light L3 to the optical fiber 12. The focal length (focal position) of the distal optical system 13 is formed, for example, at a distance of about 3 mm in the radial direction of the sheath 11 from the optical axis LP of the optical fiber 12. The measurement light L1 emitted from the distal optical system 13 is inclined by an angle of about seven degrees from a direction perpendicular to the optical axis LP.

A reflecting section 16 is provided on an outer circumference 14a of the ferrule 14. As shown in FIG. 2, the reflecting section 16 is formed by uneven surfaces 17, which are provided by a spiral rib formed on the outer circumference 14a of the ferrule 14 and are tapered at an angle θ with respect to the optical axis LP. If the angle θ is small, the amount of specular reflection light returning to the endoscope 50 is low. Therefore, specifically, the angle θ may be 40 degrees or more, or optionally be around 60 degrees, with respect to the optical axis LP, however, this is not intended to limit the invention. The reflecting section 16 may not necessarily be formed on the entire area of the outer circumference 14a, and may be formed on a part of the outer circumference 14a. The uneven surfaces are not limited to those formed by directly machining the outer circumference 14a of the ferrule 14, and may be formed by providing a machined member having the uneven surfaces 17.

Next, operation of the OCT optical probe 2 of the invention is described. As described above, the optical fiber 12 is connected to the driving means built in the proximal end portion 20. The driving means drives the optical fiber to rotate about the optical axis LP in the direction of arrow R. The rotation of the optical fiber 12 about the optical axis LP makes the distal end optical system 13, which is fixed to the optical fiber 12 via the ferrule 14, rotate about the optical axis LP in the direction of arrow R. Therefore, the OCT optical probe 2 moves the measurement light L1 emitted from the distal end optical system 13 to scan about the optical axis LP in the direction of arrow R relative to the subject to be measured M. It should be noted that this rotation is not limited to rotation in a fixed direction, and may include pivoting movement within a predetermined range.

The illumination light L5 emitted from illumination lens 65 is transmitted through the sheath 11 and is reflected at the reflecting section 16 having the uneven surfaces 17. Then, the reflected light L6 is transmitted through the sheath 11 and enters the imaging lens 66. If the reflecting section 16 is formed on a part of the outer circumference 14a of the ferrule 14, the reflected light L6 is transmitted through the sheath 11 and enters the imaging lens 66 when the uneven surfaces 17 serving as the reflecting section 16 are in a position illuminated by the illumination light L5 while the ferrule 14, which is fixed to the optical fiber 12 via an adhesive, or the like, rotates about the optical axis LP.

The uneven surfaces 17 are not limited to those formed by a protrusion which is formed on the outer circumference 14a and protrude from the diameter of the outer circumference 14a, and the uneven surfaces 17 may be formed by a depression which is formed in the outer circumference 14a and is lower than the diameter of the outer circumference 14a. Further, the uneven surfaces 17 are not limited to those formed by a spiral rib or groove, and may be formed by a plurality of ribs or grooves. Furthermore, the uneven surfaces 17 may be formed by bumps or dimples.

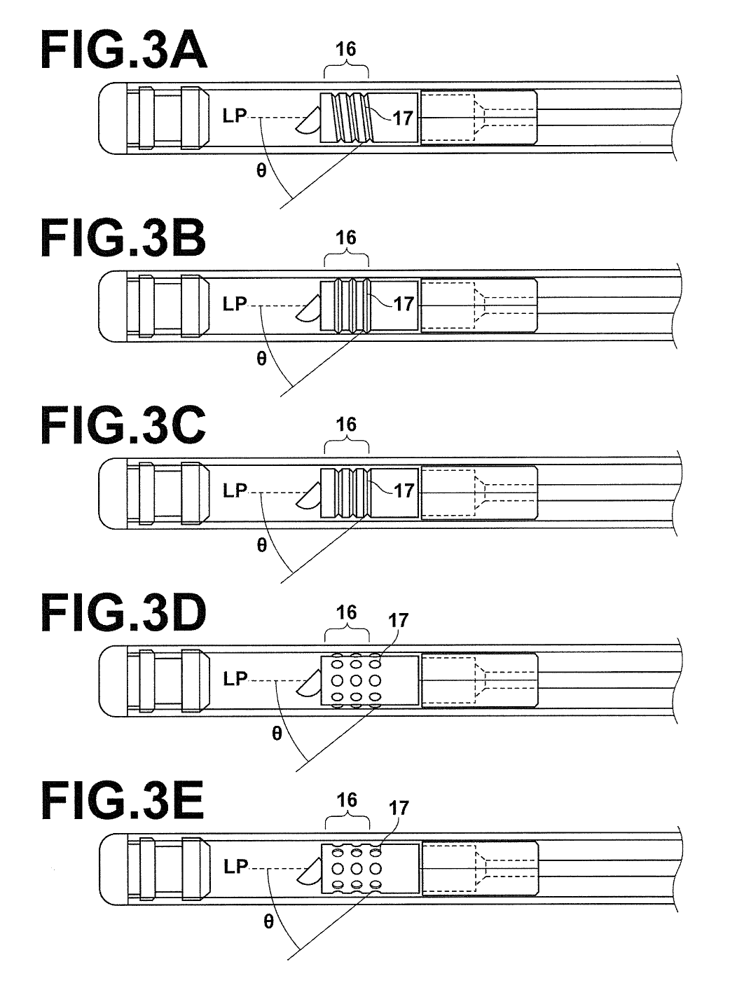

FIGS. 3A-3E illustrate further embodiments of the reflecting section 16 of the OCT optical probe 2. FIG. 3A illustrates the reflecting section 16 formed by the uneven surfaces 17 provided by a spiral groove formed in the outer circumference 14a, FIG. 3B illustrates the reflecting section 16 formed by the uneven surfaces 17 provided by a plurality of ribs formed on the outer circumference 14a, FIG. 3C illustrates the reflecting section 16 formed by the uneven surfaces 17 provided by a plurality of grooves formed in the outer circumference 14a, FIG. 3D illustrates the reflecting section 16 formed by the uneven surfaces 17 provided by a plurality of bumps formed on the outer circumference 14a, and FIG. 3E illustrates the reflecting section 16 formed by the uneven surfaces 17 provided by a plurality of dimples formed in the outer circumference 14a. Further, the uneven surfaces 17 provide by the rib(s) or groove(s) are not limited to those form the uniform angle θ. That is, the angle θ of each uneven surface 17 may be determined to concentrate the illumination light L5 on a portion of the sheath 11 to provide a difference in lightness or darkness that is clearly distinguishable from the surrounding area.

Referring again to FIG. 1, the optical tomography processing unit 100 is described. The optical tomography processing unit 100 is an optical tomography imaging apparatus using the SS-OCT measurement technique. The optical tomography imaging apparatus 100 includes: a light source unit 110 for emitting laser light L; an optical fiber coupler 101 for dividing the laser light L emitted from the light source unit 110; a period clock generator unit 120 for outputting a period clock signal T_{CLK} from the laser light divided by the optical fiber coupler 101; a light dividing section 102 for further dividing one of laser light beams divided by the optical fiber coupler 101 into the measurement light L1 and the reference light L2; an optical path length adjusting unit 130 for adjusting the optical path length of the reference light L2 divided by the light dividing section 102; a combining section 103 for combining the reference light L2 with the reflected light L3 from the OCT optical probe 2; an interference light detecting unit 140 for detecting interference light L4 formed between the reflected light L3 and the reference light L2 combined by the combining section 103; and a tomographic image processing unit 150 for applying frequency analysis to the interference light L4 detected by the interference light detecting unit 140 to carry out image processing of the image of the subject to be measured M, and outputting tomographic image signal St to the display unit 80, which will be described later.

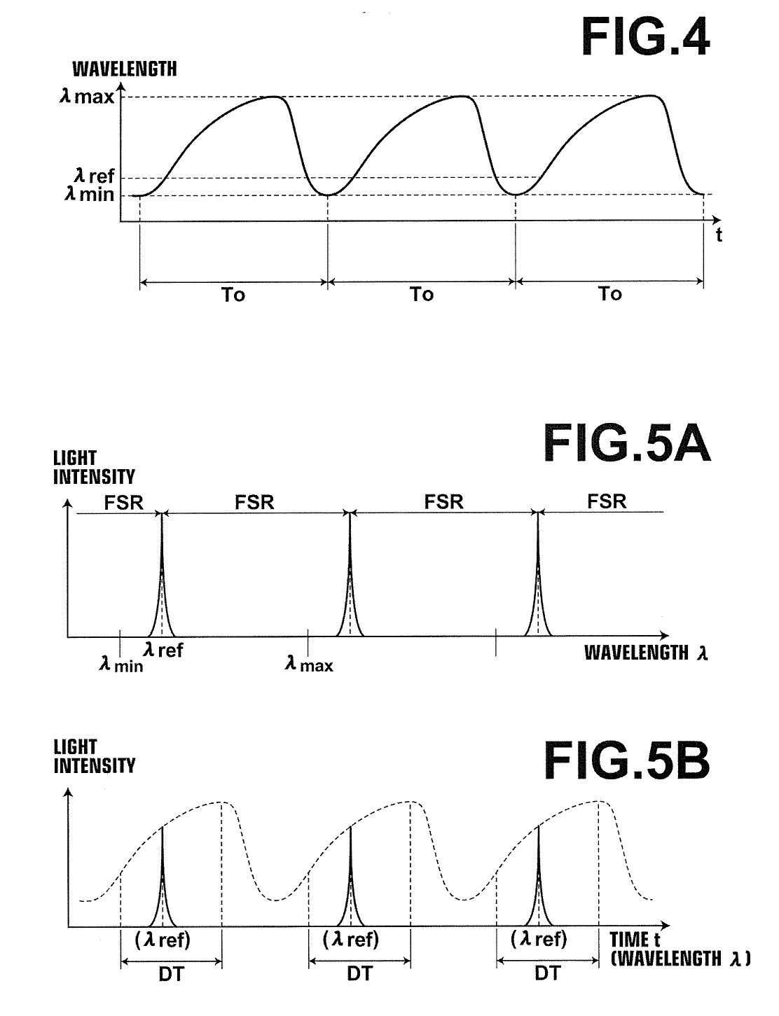

The light source unit 110 emits the laser light L with the wavelengths thereof swept in a constant period T0. Specifically, the light source unit 110 includes a semiconductor optical amplifier 111 and an optical fiber FB10 connected to opposite ends of the semiconductor optical amplifier 111. When a driving current is injected, the semiconductor optical amplifier 111 emits weak light to one end of the optical fiber FB10, and amplifies the light inputted from the other end of the optical fiber FB10. As the driving current is supplied to the semiconductor optical amplifier 111, pulsed laser light L generated by an optical resonator formed by the semiconductor optical amplifier 111 and the optical fiber FB10 is emitted to the optical fiber FB0. Further, a circulator 112 is coupled to the optical fiber FB10, so that a portion of the laser light guided through the optical fiber FB10 is emitted from the circulator 112 to an optical fiber FB11. The light emitted from the optical fiber FB11 travels through a collimator lens 113, a diffraction optical element 114 and an optical system 115, and is reflected by a rotating polygon mirror 116. The reflected laser light travels back through the optical system 115, the diffraction optical element 114 and the collimator lens 113, and re-enters the optical fiber FB11. The rotating polygon mirror 116 rotates at a high speed, such as around 30,000 rpm, in the direction of arrow R1, and the angle of each reflection facet with respect to the optical axis of the optical system 115 varies. Therefore, among the spectral components of the laser light split by the diffraction optical element 114, only the component of a particular wavelength range returns to the optical fiber FB11. Then, the laser light of the particular wavelength range entering the optical fiber FB11 is inputted via the circulator 112 to the optical fiber FB10. As a result, the laser light L of the particular wavelength range is emitted to the optical fiber FB0. Therefore, when the rotating polygon mirror 116 rotates at a constant speed in the direction of arrow R1, the wavelength λ of the laser light re-entering the optical fiber FB11 varies with time in a constant period. As shown in FIG. 4, the light source unit 110 emits the laser light L with the wavelength thereof swept from a minimum sweep wavelength λmin to a maximum sweep wavelength λmax in a constant period T0 (for example, about 50 μsec). The wavelength-swept laser light L is emitted to the optical fiber FB0.

The optical fiber coupler 101 divides and directs the laser light L inputted to the optical fiber FB0 to the optical fibers FB1 and FB5. The laser light L emitted to the optical fiber FB5 is guided to the period clock generator unit 120. The laser light emitted to the optical fiber FB1 is guided to the light dividing section 102.

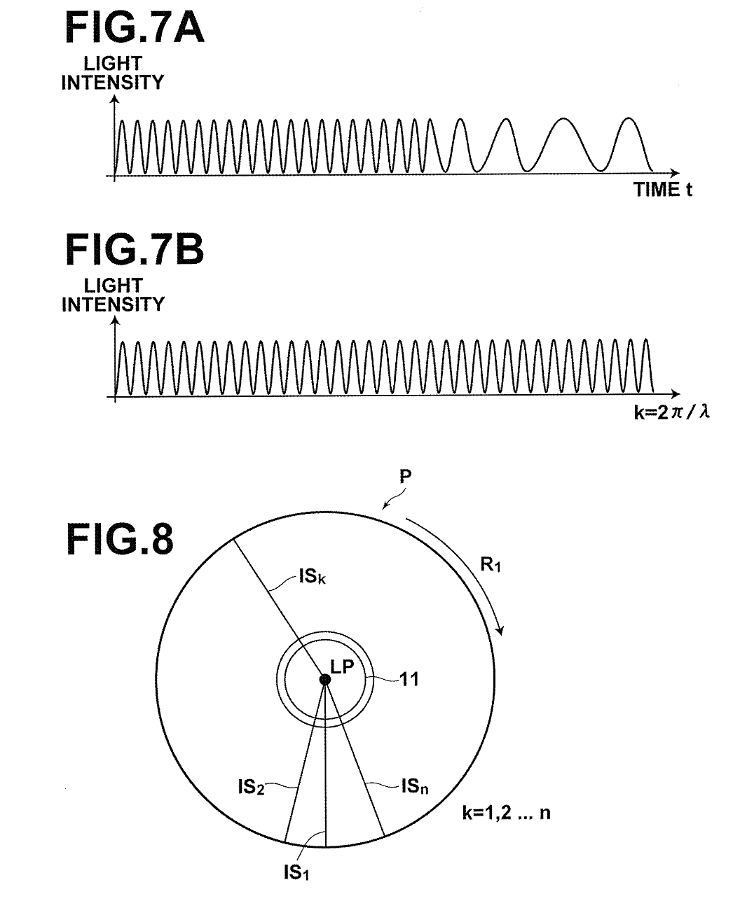

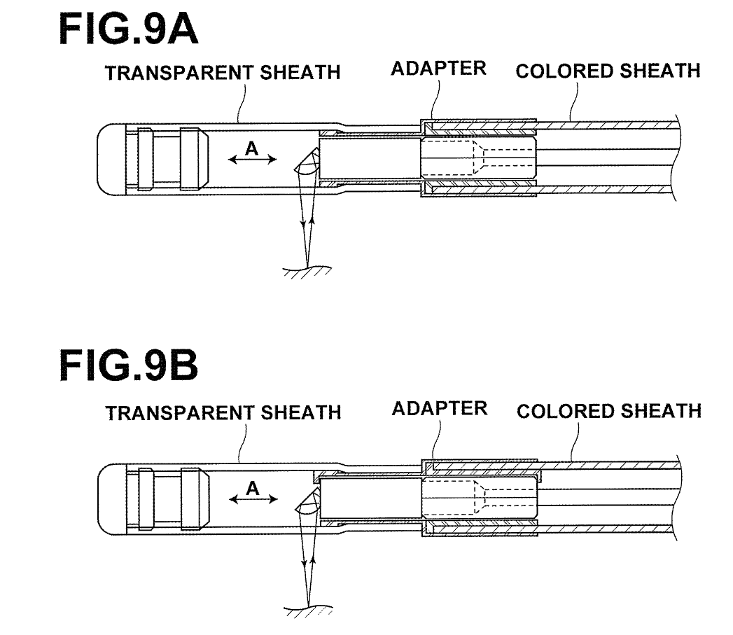

The period clock generator unit 120 outputs the period clock signal T_{CLK} each time the wavelength of the laser light L emitted from the light source unit 110 is swept over one period. The period clock generator unit 120 includes optical lenses 121 and 123, an optical filter 122 and a photodetector unit 124. The laser light L emitted from the optical fiber FB5 enters the optical filter 122 via the optical lens 121. The laser light L transmitted through the optical filter 122 is then detected by the photodetector unit 124 via the optical lens 123, and the period clock signal T_{CLK} is outputted to the tomographic image processing unit 150. As shown in FIG. 5A, the optical filter 122 transmits only the laser light L having a set wavelength λref, and blocks the laser light L of other wavelength bands. The optical filter 122 has a FSR (free spectrum range), which is a light transmission period in which one of plurality of transmission wavelengths is set within the wavelength band of λmin-λmax. Therefore, only the laser light L having the set wavelength λref within the wavelength band of λmin-λmax, within which the wavelength of the laser light L emitted from the light source unit 110 is swept, is transmitted through the optical filter 122, and the laser light L of other wavelength bands is blocked. As shown in FIG. 7B, the period clock signal T_{CLK} is outputted when the wavelength of the laser light L with the periodically swept wavelength emitted from the light source unit 110 is the set wavelength λref.

The light dividing section 102 divides the laser light L guided to the optical fiber FB1 into the measurement light L1 and the reference light L2. The measurement light L1 is guided through the optical fiber FB2, and the reference light L2 is guided through the optical fiber FB3 to enter the optical path length adjusting unit 130. The optical fiber FB2 is optically connected to the optical fiber 12. It should be noted that the light dividing section 102 in this embodiment also serves as the combining section 103.

The optical path length adjusting unit 130 changes the optical path length of the reference light L2 to adjust the position at which acquisition of the tomographic image is started. The optical path length adjusting unit 130 includes: a reflection mirror 132 for reflecting the reference light L2 emitted from the optical fiber FB3; a first optical lens 131a disposed between the reflection mirror 132 and the optical fiber FB3; and a second optical lens 131b disposed between the first optical lens 131a and the reflection mirror 132. The reference light L2 emitted from the optical fiber FB3 is collimated by the first optical lens 131a and is collected by the second optical lens 131b onto the reflection mirror 132. Then, the reference light L2 reflected from the reflection mirror 132 is collimated by the second optical lens 131b and is collected by the first optical lens 131a onto the optical fiber FB3. The optical path length adjusting unit 130 further includes: a base 133, on which the second optical lens 131b and the reflection mirror 132 are fixed; and a mirror moving means 134 for moving the base 133 along the optical axis of the first optical lens 131a. The optical path length of the reference light L2 is changed by moving the base 133 in the direction of arrow A.

The combining section 103 combines the reflected light L3 from the subject to be measured M with the reference light L2 having the optical path length adjusted by the optical path length adjusting unit 130, and emits the interference light L4 to the interference light detecting unit 140 via the optical fiber FB4.

The interference light detecting unit 140 detects the interference light L4 and outputs an interference signal IS. It should be noted that, in this apparatus, the interference light L4 is divided into two parts by the light dividing section 102 and these parts are guided to the photodetectors 140a and 140b to be calculated, so that balanced detection is carried out. The interference signal IS is outputted to the tomographic image processing unit 150.

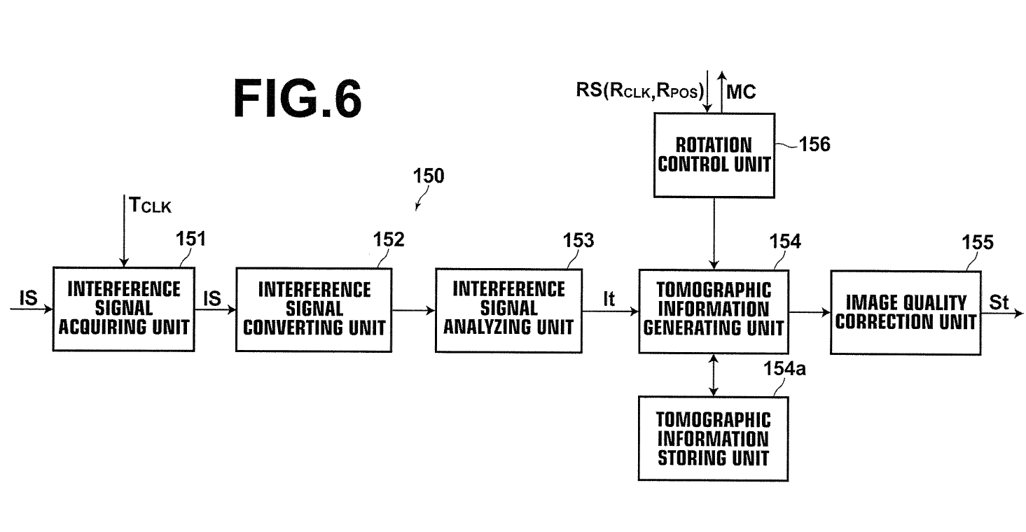

FIG. 6 is a block diagram illustrating the schematic configuration of the tomographic image processing unit 150. The tomographic image processing unit 150 outputs the tomographic image signal St based on the interference signal IS. The tomographic image processing unit 150 includes an interference signal acquiring unit 151, an interference signal converting unit 152, an interference signal analyzing unit 153, a tomographic image information generating unit 154, an image quality correction unit 155 and a rotation control unit 156.

The interference signal acquiring unit 151 acquires the interference signal IS for one period, which is detected by the interference light detecting unit 140, based on the period clock signal T_{CLK} outputted from the period clock generator unit 120. The interference signal acquiring unit 151 acquires the interference signal IS of a wavelength band DT (see FIG. 5B) spanning between points before and after the output timing of the period clock signal T_{CLK}.

The interference signal converting unit 152 rearranges the interference signal IS acquired by the interference signal acquiring unit 151 in equal intervals along the wavenumber k(=2π/λ) axis. FIG. 7A illustrates the interference signal IS. FIG. 7B illustrates the rearranged interference signal IS. Specifically, the interference signal converting unit 152 is provided in advance with a time-wavelength sweep characteristics data table or function of the light source unit 110, and uses this time-wavelength sweep characteristics data table or function to rearrange the interference signal IS in equal intervals along the wavenumber k axis.

The interference signal analyzing unit 153 acquires the tomographic information It by applying a known spectral analysis technique, such as the Fourier transformation, the maximum entropy method, or the like, to the interference signal IS converted by the interference signal converting unit 152.

The rotation control unit 156 controls the driving means built in the proximal end portion 20 of the OCT optical probe 2. Specifically, the rotation control unit 156 outputs a control signal MC to a driving source, such as a motor, of the driving means, and receives the rotation signal RS inputted from an encoder, or the like, of the driving means. The rotational position signal RS includes a rotation clock signal R_{CLK}, which is generated for each rotation of the driving source, and a rotational angle signal Rₚₒₛ.

The tomographic information generating unit 154 acquires the tomographic information It, which corresponds to scanning in the radial direction by the distal end portion 10 of the OCT optical probe 2, for one period (one line) acquired by the interference signal analyzing unit 153. FIG. 8 schematically illustrates an operation carried out by the tomographic information generating unit 154. The tomographic information generating unit 154 stores the tomographic information It for one line, which is sequentially acquired, in a tomographic information storing unit 154a. The tomographic information generating unit 154 can generate the tomographic information It corresponding to the radial scan by reading the tomographic information It for n lines at a time from the tomographic information storing unit 154a based on the rotation clock signal R_{CLK} inputted to the rotation control unit 156. Alternatively, the tomographic information generating unit 154 can generate the tomographic information It corresponding to the radial scan by sequentially reading the tomographic information It from the tomographic information storing unit 154a based on the rotational angle signal Rₚₒₛ inputted to the rotation control unit 156.

The image quality correction unit 155 applies correction, such as sharpness correction and smoothness correction, to the tomographic information It inputted from the tomographic information generating unit 154, and outputs the tomographic image signal St to the display unit 80.

The display unit 80 includes an observation monitor 81 and a tomography monitor 82. The observation monitor 81 receives the observation image signal So inputted from the video processor 72 of the endoscope 50 and displays an observation image Po. The tomography monitor 82 receives the tomographic image signal St inputted from the tomographic image processing unit 150 of the optical tomography processing unit 100 and displays an optical tomographic image Pt.

Next, operation of a specific embodiment of the invention is described. The operator inserts the inserted portion 60 of the endoscope 50 into the body cavity B of the subject. The illumination light L5 from the illumination light source 71 enters the illumination lens 65 via the light guide 63 to illuminate the site to be observed O in the body cavity B. The reflected light L6 from the site to be observed O illuminated by the illumination light L5 enters the imaging lens 66 and is reflected by the prism 67 to enter the CCD image pickup device 64. The observation image information Io obtained through photoelectric conversion at the CCD image pickup device 64 is inputted to the video processor 72 via the CCD cable 62. The video processor 72 carries out image processing and outputs the observation image signal So, and the observation image Po is displayed on the observation monitor 81.

The operator inserts the OCT optical probe 2 through the forceps channel 61 so that the OCT optical probe 2 extends from the distal end of the inserted portion 60 of the endoscope 50 and is inserted into the body cavity B. The illumination light L5 from the illumination lens 65 is directed to the OCT optical probe 2 inserted into the body cavity B. The illumination light L5 is transmitted through the sheath 11 and is reflected at the reflecting section 16, which is formed on the outer circumference 14a of the ferrule 14, toward the imaging lens 66. The reflecting section 16 is displayed on the observation monitor 81 as a bright portion Bp. The operator views the bright portion Bp of the reflecting section 16 displayed on the observation monitor 81 to confirm the scanning position SC of the measurement light L1 of the OCT optical probe 2 on the subject to be measured M. As described above, if the reflecting section 16 is provided on a part of the outer circumference 14a of the ferrule 14, the bright portion Bp is displayed on the observation monitor 81 when the reflecting section 16 is in a position where the illumination light L5 directed to the reflecting section 16 is reflected toward the imaging lens 66 as the reflected light L6, while the driving means built in the proximal end portion 20 of the OCT optical probe 2 drives the optical fiber 12 to rotate about the optical axis LP. That is, the operator confirms the scanning position SC as the bright portion Bp blinking on the observation monitor 81.

The operator confirms the scanning position SC of the OCT optical probe 2 based on the bright portion Bp, and moves the distal end portion 10 of the OCT optical probe 2 so that the scanning position SC is set in a desired position on the subject to be measured M. The laser light L from the light source unit 110 of the optical tomography processing unit 100 is divided at the optical fiber coupler 101. One of the divided laser beams is inputted to the period clock generator unit 120 and the period clock signal T_{CLK} is generated. The other of the laser beams is inputted to the light dividing section 102 and is divided into the measurement light L1 and the reference light L2. The reference light L2 enters the optical path length adjusting unit 130 and the optical path length is adjusted. The measurement light L1 is emitted from the distal end optical system 13 of the OCT optical probe 2 via the optical fiber 12 toward the subject to be measured M, and the reflected light L3 from the subject to be measured M re-enters the distal end optical system 13. The driving means built in the proximal end portion 20 of the OCT optical probe 2 drives the optical fiber 12 to rotate about the optical axis LP in the direction of arrow R to effect scanning about the optical axis LP. The reflected light L3 re-enters the optical fiber 12 and is inputted to the combining section 103, where the interference light L4 is generated between the reflected light L3 and the reference light L2 having the optical path length thereof adjusted. The interference light L4 is then inputted to the interference light detecting unit 140 and the interference signal IS is generated. The tomographic image processing unit 150 generates the tomographic image signal St based on the interference signal IS. The tomographic image signal St is inputted to the tomography monitor 82 and the optical tomographic image Pt is displayed on the tomography monitor 82.

In the OCT optical probe 2 of the invention, the reflecting section 16 provided at the outer circumference 14a of the ferrule 14 reflects the illumination light L5 applied from the endoscope 50 back to the endoscope 50, thereby allowing the operator to visually confirm the scanning position SC based on the bright portion Bp in the observation image Po.

As described above, the OCT optical probe 2 of the invention does not use a colorant, or the like, in the sheath for visual confirmation of the scanning position SC, and therefore is free of the problem of biocompatibility of the colorant. Further, since it is not necessary to superimpose the aiming light on the measurement light L1, there is no cost increase.

Thus, the OCT optical probe 2 of the invention can provide safe and inexpensive visual confirmation of the scanning position SC.

The optical tomography imaging apparatus 1 according to the invention, to which the above-described OCT optical probe 2 is applied, can also provide safe and inexpensive visual confirmation of the scanning position SC.

Although the optical tomography processing unit 100, to which the OCT optical probe 2 of the invention is applied, has been described as an SS-OCT apparatus in the above-described embodiment by way of example, the OCT optical probe 2 of the invention is also applicable to SD-OCT and TD-OCT apparatuses.

According to the OCT optical probe of the invention, a part of the illumination light is reflected at the reflecting section, which is formed at the outer circumference of the ferrule, toward the endoscope, so that the position of the reflecting section is imaged to allow visual confirmation of the scanning position of the OCT optical probe based on the position of the reflecting section. That is, the OCT optical probe of the invention does not require consideration of biocompatibility of a colorant applied to the colored sheath of prior art, and thus is safe. Further, even when the OCT optical probe is flexed during operation, the distance between the reflecting section and the scanning position is kept constant, and therefore the operator can visually confirm the scanning position in a stable manner. In addition, the ferrule can freely move in the sheath along the longitudinal direction thereof, and therefore the optical fiber is not stressed. Moreover, it is not necessary to superimpose the aiming light on the measurement light L1, and therefore there is no cost increase.

Thus, the OCT optical probe of the invention can provide safe and inexpensive visual confirmation of the scanning position of the OCT optical probe using the endoscope.

The optical tomography imaging apparatus according to the invention, to which the above-described OCT optical probe is applied, can also provide safe and inexpensive visual confirmation of the scanning position of the OCT optical probe using the endoscope.

What is claimed is:

1. An OCT optical probe to be inserted into a forceps channel of an endoscope that applies illumination light to a site to be observed in a body cavity and images reflected light from the site to be observed, the OCT optical probe comprising:
a substantially cylindrical and long sheath to be inserted into the body cavity;
a distal end optical system disposed in the sheath and being rotatable about the longitudinal axis of the sheath; and
a ferrule integrally fixed to the distal end optical system, wherein the ferrule comprises a reflecting section adapted to reflect a part of the illumination light toward the endoscope.

2. The OCT optical probe as claimed in claim 1, wherein the reflecting section is formed on a partial or entire area of an outer circumference of the ferrule.

3. The OCT optical probe as claimed in claim 2, wherein the reflecting section is formed by uneven surfaces provided on the outer circumference of the ferrule.

4. An optical tomography imaging apparatus comprising:
a light source unit for emitting light;
a light dividing section for dividing the light emitted from the light source unit into measurement light and reference light;
an OCT optical probe for applying the measurement light to a subject to be measured;
a combining section for combining the reference light with reflected light from the subject to be measured when the measurement light is applied to the subject to be measured;
an interference light detecting unit for detecting interference light formed between the combined reflected light and reference light; and
a tomographic image processing unit for detecting reflection intensity at a plurality of depth positions in the subject to be measured based on frequency and intensity of the detected interference light, and acquiring an optical tomographic image of the subject to be measured based on the intensity of the reflected light at each depth position,
wherein the OCT optical probe comprises the OCT optical probe as claimed in claim 1.
